(19) United States
(12) Patent Application Publication
(10) Pub. No.: US 2012/0093802 A1
(43) Pub. Date: Apr. 19, 2012

(54) BIOACTIVE COMPOSITION INCLUDING STABILIZED PROTEIN AND PROCESS FOR PRODUCING THE SAME

(75) Inventors: Ping Wang, North Oaks, MN (US); Songtao Wu, St. Paul, MN (US); Hongfei Jia, Ann Arbor, MI (US); Masahiko Ishii, Okazaki City (JP); Minjuan Zhang, Ann Arbor, MI (US)

(73) Assignee: Toyota Motor Engineering & Manufacturing North America, Inc., Erlanger, KY (US)

(21) Appl. No.: 12/903,312

(22) Filed: Oct. 13, 2010

(51) Int. Cl. A61K 38/48; A61K 38/47; A61K 38/46; A61K 38/44; A61P 37/08; A61K 39/395; A61K 31/7105; A61K 38/22; A61K 31/727; A61K 31/715; A61K 38/51

(52) U.S. Cl. 424/130.1; 514/57; 424/94.61; 424/94.63; 424/94.6; 424/94.4; 424/94.5; 514/44 R; 514/9.7; 514/56

(57) ABSTRACT

A bioactive composition includes a porous hydrogel matrix. At least one protein is immobilized in the porous hydrogel matrix forming a hydrogel protein composite that is stable in an organic solvent. A process for stabilizing a bioactive composition includes the steps of: forming hydrogel matrix pores around protein molecules and reducing a water content within the hydrogel matrix pores forming a hydrogel protein composite that is stable in an organic solvent.

### FIELD OF THE INVENTION

The invention relates to compositions and processes for stabilizing bioactive materials.

### BACKGROUND OF THE INVENTION

Bioactive macromolecules such as proteins, nucleic acids, and functional enzymes may be utilized in various aspects of biomedical and industrial applications. For example, nucleic acids may be utilized as genetic templates for polymerase chain reactions while proteins may be utilized in various detergent mixtures to enhance digestive cleaning efficiency of the detergent. Additionally, proteins such as digestive proteins or enzymes may be utilized to catalyze and decompose organic molecules. Digestive proteins may be utilized in organic media allowing various substrates to be utilized. Should the substrate be insoluble or only soluble in water, the maximum activity of the digestive proteins may not be achieved in an aqueous solution.

Although proteins such as digestive proteins or enzymes may be capable of decomposing and reacting with various organic molecules, they are generally not thermally stable at elevated temperatures. Additionally, such proteins or digestive enzymes are generally not stable in a non-aqueous organic solvent.

There is therefore a need in the art for a thermally stable bioactive composition that may be utilized in elevated temperatures and under dry conditions. There is also a need in the art for a bioactive composition that maintains a high catalytic activity in an organic solvent. There is additionally a need in the art for a thermally stable bioactive composition that maintains a high catalytic activity in organic solvent and a process for producing the bioactive composition.

### SUMMARY OF THE INVENTION

In one aspect, there is disclosed a bioactive composition including a porous hydrogel matrix. At least one protein that is immobilized in the porous hydrogel matrix forming a hydrogel protein composite that is stable in an organic solvent.

In another aspect, there is disclosed a process for stabilizing a bioactive composition that includes the steps of: forming hydrogel matrix pores around protein molecules and reducing a water content within the hydrogel matrix pores forming a hydrogel protein composite that is stable in an organic solvent.

### BRIEF DESCRIPTION OF THE DRAWINGS

FIG. 1 is a graph of the relative activity as a function of time for a pretreated gel-entrapped GOₓ as well a nontreated gel-entrapped GOₓ and a native GOₓ enzyme in ethanol;

FIG. 2 is a plot of molecular weight of an enzyme as a function of monomer concentration to provide entrapment in a hydrogel;

FIG. 3 is a graph of the relative activity as a function of time at 80° centigrade for a pretreated α-chymotrypsin after incubation at 80° centigrade for different time periods;

FIG. 4 is a plot of the relative activity as a function of time in methanol for a native enzyme of α-CT, a nontreated hydrogel enzyme of α-CT, and a pretreated hydrogel enzyme of α-CT;

FIG. 5 is a plot of the reaction rate of a transesterification reaction for both a treated gel α-CT and a native enzyme of α-CT;

FIG. 6 is a plot detailing Initial transesterification rate for the native and the dry-hydrogel entrapped α-Chymotrypsin with dependence on different water content;

FIG. 7 is a plot of the relative activity as a function of time for a native α-CT enzyme and a hydrogel entrapped α-CT enzyme for a transesterification reaction in n-hexane;

FIG. 8 is a plot of the relative activity of a gel confined enzyme over various cycles of use in both hydrolytic and organic transesterification reactions;

### DETAILED DESCRIPTION OF THE PREFERRED EMBODIMENTS

There is disclosed herein a bioactive composition that includes a porous hydrogel matrix and at least one protein immobilized in the porous hydrogel matrix that forms a hydrogel protein composite that is stable in organic solvent and at elevated temperatures. In one aspect, the hydrogel protein composite has a half-life that is at least 1000 times longer than the half-life of a free digestive protein counterpart in the organic solvent. Additionally, the hydrogel protein composite maintains an activity in the organic solvent. In one aspect, the hydrogel protein composite has an activity that is 1000 times greater than a free digestive protein counterpart in the organic solvent.

In addition to the stability of the hydrogel protein composite in an organic solvent, the hydrogel protein remains biologically active at elevated temperatures. In one aspect, the protein remains biologically active after exposure to a temperature of up to 100° Celsius.

Various proteins may be utilized in the bioactive composition disclosed herein. In one aspect, the protein may be selected from the group consisting of protease, amylase, cellulose, lipase, peroxidase, tyrosinase, glycosidase, nuclease, aldolase, phosphatase, sufatase, dehydrogenase, and lysozyme and combinations thereof. Additionally, bioactive agents may be utilized including antibodies, nucleic acids, fatty acids, hormones, vitamins, minerals, structural proteins, enzymes, and therapeutic agents including histamine blockers and heparin.

The bioactive composition includes a hydrogel matrix that has a water content of from 0 to 0.5 weight percent of the hydrogel matrix. In one aspect, the digestive protein is between 0.1 to 10 dry weight percent of the hydrogel matrix and in one aspect from 0.2 to 4.5 dry weight percent of the hydrogel matrix.

There is also disclosed herein a process for stabilizing a bioactive composition that includes forming a hydrogel matrix pore around a protein molecule and reducing a water content within the hydrogel matrix pores forming a hydrogel protein composite that is stable in organic solvent. In one aspect, the step of forming the hydrogel matrix pores around the protein molecules includes the step of dissolving a protein in deionized water at a desired concentration and dissolving a prepolymer in deionized water at a desired concentration and mixing the dissolved protein and dissolved prepolymer in a specified ratio. Next, polymerization is initiated of the prepolymer composition. In one aspect, the step of initiating the polymerization includes at least one step that may be selected from adding a cross linking agent, adding an initiator, adjusting a temperature of the mixture of the dissolved protein and the dissolved prepolymer. In one aspect, the ratio of dissolved prepolymer is from 20 to 28 percent by weight in relation to the total volume of the prepolymer.

The step of reducing a water content within the hydrogel matrix pores may include heating the hydrogel matrix to a temperature of from 20 to 100 degrees Celsius for a time period of from 24 hours to 7 days. Additionally, the step of reducing a water content within the hydrogel matrix may include heating the hydrogel matrix to a temperature of from 20 to 80 degrees Celsius for a time period of 24 hours followed by air drying at room temperature for a specified period such as 1 week.

Additionally, the step of reducing a water content within the hydrogel matrix pores may include heating the hydrogel matrix to a temperature of from 20 to 55 degrees Celsius for a time period of 24 hours followed by air drying at room temperature for a specified time period such as 1 week.

Further, the step of reducing a water content within the hydrogel matrix pores may reduce the pore volumes of from 15 to 21 percent compared to a wet gel volume. The step of reducing water content may reduce the pore volumes and allow a substrate to enter the pores allowing a reaction between the protein and a substrate. In this manner, the bioactive composition may be utilized in various reactions. In one aspect, the cross linking of the bioactive composition may be adjusted to again allow access of a substrate into the pores of the entrapped enzyme. The bioactive composition may have various shapes such as fibers, beads, filaments, or rods when used for various reactions.

In one aspect, the pretreatment of the bioactive composition may vary based on a transition temperature that would trigger a structural change of the enzyme being utilized. Referring to the table presented below, it can be seen that various enzymes have transition temperatures labeled TM in degrees Celsius. Listed in the table are various enzymes including glucose oxidase, peroxidase, α-chymotrypsin, thermolysin, α-amylase, and lipase. As can be seen in the table, the suggested pretreatment temperatures will vary based upon the transition temperature TM. As can be seen in the table, the suggested pretreatment temperature does not greatly exceed the transition temperature which would result in a large structural change of an enzyme.

| | | | | | | Suggested |
|---|---|---|---|---|---|---|
| | | | | Tm | | pretreament T |
| # | Enzyme | EC # | Source | (° C.) | Ref | (° C.) |
| 1 | gluocose oxidase | 1.1.3.4 | Aspergillus niger | 62 | 1 | 80 |
| 2 | peroxidase | 1.11.1.7 | Horse radish | 42 | 2 | 50 |
| 3 | α-chymotrypsin | 3.4.21.1 | Bovine pancreas | 44 | 3 | 55 |
| 4 | thermolysin | 3.4.24.4 | Bacillus | 87 | 4 | 99 |
| | | | thermopmfeolyticus | | | |
| 5 | α-amylase | 3.2.1.1 | Bacillus | 60 | 5 | 76 |
| | | | amyloliquefaciens | | | |
| 6 | lipase | 3.1.1.3 | Candida cylindracea | 86 | 6 | 99 |

Various aspects of the present invention are illustrated by the following nonlimiting examples. The examples are for illustrative purposes and are not a limitation on the practice of the present invention. It will be understood that variations and modifications can be made without departing from the spirit and scope of the invention.

### EXAMPLES

### Example 1

### Entrapment of Glucose Oxidase into Polyacrylamide Hydrogel with Enhanced Thermo and Catalytic Stability

Materials

Acylamide/Bis solution and NNN′N′-tetramethyleethylenediamine (TEMED) were the products of Bio-Rad Laboratories, Hercules, Calif., USA. D-(+)-glucose, glucose oxidase (GOₓ) from aspergillus niger (EC 1.1.3.4), peroxidase (HRP) from horseradish (EC 1.11.1.7), o-dianisidine, HPLC grade ethanol, methanol and chloroform, toluene, ammonium persulfate were obtained from Sigma Chemical Co., St. Louis, Mo., USA. Unless specially mentioned, all other reagents and solvents used in the experiments were of the highest grade commercially available.

Entrapment of Glucose Oxidase (GOₓ) into Polyacrylamide Hydrogel

The entrapment of GOₓ into polyacrylamide hydrogel was performed by the following procedure: 2 ml of 0.1M pH 7.0 sodium phosphate buffer containing 0.5-10 mg GOₓ were prepared and subsequently mixed with 6.8 ml of 30% acylamide/bis solution and 1.2 ml of DI H₂O to make a 10 ml of solution with total monomers concentration of 20% (w/v) and cross-linker concentration of 5% (w/w). The polymerization using enclosures (8.3×7×0.075 cm³) to obtain hydrogel disks of predefined shape was initiated by adding 100 μl fresh prepared ammonium persulfate (10% w/v in DI water) and 4 μl of TEMED at room temperature. A period of at least 4 hours was needed for complete gelling to entrap the enzyme. The resulting hydrogel disks were removed from the glass enclosures and punched into small disks with a diameter of 16 mm for further tests.

Activity Assays for Native and Hydrogel-Entrapped GOₓ

A coupled-enzyme reaction using horseradish peroxidase and o-dianisidine was applied to determine the activity of GOₓ. For the native enzyme, the reaction mixture (1.1 ml) contained 0.1 mol glucose, 7 μg horseradish peroxidase, 0.17 mM o-dianisidine and 35 μl enzyme (0.4-0.8 U/ml) in 50 mM pH 5.1 sodium acetate buffer. The increase in absorbance at 500 nm at room temperature was recorded for activity calculation. The reaction with hydrogel-entrapped GOₓ was conducted in 20-ml glass vials.

To measure the activity of hydrogel-entrapped GOₓ, the dried hydrogel disc was immersed into DI water for at least 2 hours to reach the fully swollen state before activity test. The hydrogel disc was added to 20.7 ml of 0.1M glucose solution containing 0.14 mg horseradish peroxidase and 1.1 mg o-dianisidine. Aliquots of 1 ml each were taken periodically and recombined immediately after measuring the product concentration using UV absorbance at 500 nm.

Pretreatment (Drying) of Hydrogel-Entrapped Enzymes

For hydrogel-entrapped glucose oxidase, the effective pretreated temperature was found to be in the range from 20° C. to 80° C. Preferably, the fresh hydrogel discs were placed into a petri dish and incubated in the oven at 80° C. for 24 hours, followed with drying in the air at room temperature for 1 week allowing for complete desiccation. Finally the dry hydrogel discs were used for further testing and the activity was measured by hydrolysis in an aqueous solution as described above.

Thermal Stability of Pretreated Hydrogel-Entrapped GOₓ

GOₓ containing hydrogel discs were pretreated as stated above, placed on a glass plate and incubated in an oven at high temperature (80, 110, and 130° C.). At certain time periods hydrogel discs were withdrawn from the oven and residual catalytic activity was assessed using the assay procedure as described above.

### Stability in Organic Solvents of Pretreated Hydrogel-Entrapped GOₓ

After pretreatment, the stability of hydrogel-entrapped enzymes was investigated in organic solvent such as polar type like acetone, methanol, ethanol or nonpolar type such as toluene. The pretreated hydrogel discs from the same batch were incubated in 10 ml of each solvent in screw-capped 20-mL vials. The native enzyme and non-pretreated hydrogel discs served as comparisons. The hydrogel discs were removed from the solvent at specific time periods for activity assay to determine the residual activity.

It was found that at room temperature the average lifetime of the pretreated hydrogel-entrapped-GOₓ in methanol was estimated as long as 5,650 hours whereas that of the native enzyme was less than 5 minutes and it was 50.3 hours for non pretreated wet hydrogel entrapped enzyme.

TABLE 1
Stability of native and gel-confined enzymes in organic solvents. Temperature was
controlled at 21° C.

| | Solvent | | Non | |
|---|---|---|---|---|
| | (log P) | Native GOₓ | pretreated | Pretreated |
| Half-life (h) | Methanol (−0.76) | 0.05 | 50.3 | 5650 |
| Stability enhancement | | | ~10³ | ~10⁵ |
| comparing with native enzyme | | | | |
| Half-life (h) | Acetone (−0.23) | 0.26 | 73.9 | — |
| Stability enhancement | | | 284 | — |
| comparing with native enzyme | | | | |
| Half-life (h) | Ethanol (−0.24) | 0.1 | 182 | 10260 |
| Stability enhancement | | | ~2 × 10³ | ~10⁵ |
| comparing with native enzyme | | | | |

Stability of Dried Hydrogel-Entrapped GOₓ in Organic Solvent at High Temperature

After pretreatment, the stability of hydrogel-entrapped GOₓ was investigated under the concomitant impact of high temperature and a polar solvent. Typically, the dried hydrogel discs were incubated in 10 ml of pure ethanol at a fixed temperature of 74° C. Screw-capped 20-mL vials were used. At certain times the hydrogel discs were removed from ethanol solution to determine the residual activity.

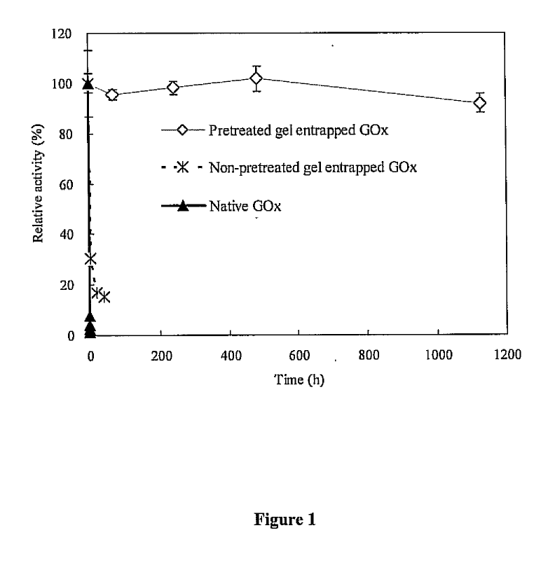

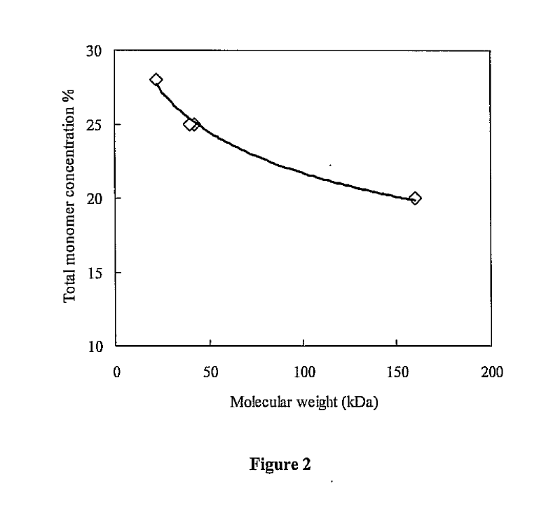

As detailed in FIG. 1 there ist no significant loss of activity for pretreated shrunk hydrogel entrapped GOₓ over a long time, whereas both the native and non pretreated wet hydrogel entrapped GOₓ were rapidly inactivated. Within the chosen incubation time of 1150 hours only 25% of the initial activity was lost. The half-life was estimated to be in the range of 4500 hours, equaling an astonishing enhancement of 2×10⁶ folds in stability compared with native enzyme under the same conditions.

### Example 2

### Entrapment of α-Chymotrypsin into Polyacrylamide Hydrogel with Enhanced Thermo and Catalytic Stability

Materials

Many of the same materials detailed in example 1 were used with the further use of, α-chymotrypsin (α-CT) from bovine pancreas (EC 3.4.21.1), n-acetyl-L-phenylalanine ethyl ester (APEE), dimethylsulfoxide (DMSO) and n-succinyl-ala-ala-pro-phe (SAAPPN) purchased from Sigma-Aldrich (St. Louis, Mo., USA). n-Propyl alcohol (n-PrOH, HPLC grade) were purchased from EM (Gibbstown, N.J.). All organic solvents were treated with 3 Å molecular thieve for at least 24 hours before being used. Unless specially mentioned, all other reagents and solvents used in the experiments were of the highest grade commercially available.

Entrapment of α-Chymotrypsin (α-CT) into Polyacrylamide Hydrogel

The entrapment of α-CT into polyacrylamide hydrogel was performed by the following procedure: 0.42 ml of 0.01 M pH 7.5 sodium acetate buffer containing 0.5-10 mg α-CT were prepared and subsequently mixed with 9.33 ml of 30% acylamide/bis solution and 0.25 ml of DI H₂O to make a 10 ml of solution with total monomers concentration 28% w/v % and cross-linker concentration 5%. The polymerization using glass enclosures to obtain hydrogel disks of predefined shape (8.3×7×0.075 cm³) was initiated by adding 100 μl fresh prepared ammonium persulfate (10% w/v in DI water) and 4 μl of TEMED at room temperature. A period of at least 4 hours was needed for complete gelling to entrap enzyme. The resulting hydrogel disk was removed from the glass enclosures and punched into small discs with a diameter of 16 mm for further tests.

Aqueous Activity Assays for Native and Hydrogel-Entrapped α-CT

For native enzyme, 50 μl of enzyme solution (1 mg/ml) were mixed with 2.44 ml of SAB and 13 μl of 160 mM SAAPPN stock solution in DMSO. The reaction rates were determined by monitoring the absorbance at 410 nm.

For hydrogel-entrapped α-CT, the dried gel disc was immersed into DI water for at least 2 hours to reach the fully swollen state before activity test. The activity was measured through the reaction in 20-ml vials with 4.975 ml of pH 7.5, 10 mM sodium acetate buffer with 5 mM calcium acetate and 25 μl of 160 mM SAAPPN stock. The reaction was initiated by the addition of hydrogel-entrapped enzyme with stirring at 200 rpm. Aliquots of 1 ml each were taken periodically and recombined immediately after measuring the product concentration using UV absorbance at 410 nm.

Pretreatment (Drying) of Hydrogel-Entrapped Enzymes

For hydrogel-entrapped α-chymotrypsin, the effective temperature for drying was found to be in the range from 20° C. to 55° C. Preferably, the fresh hydrogel discs were incubated in the oven at 55° C. for 24 hours followed by drying in the air at room temperature for 1 week allowing for complete desiccation. Finally the dry hydrogel discs were used for further testing and the activity was measured by hydrolysis in an aqueous as described above.

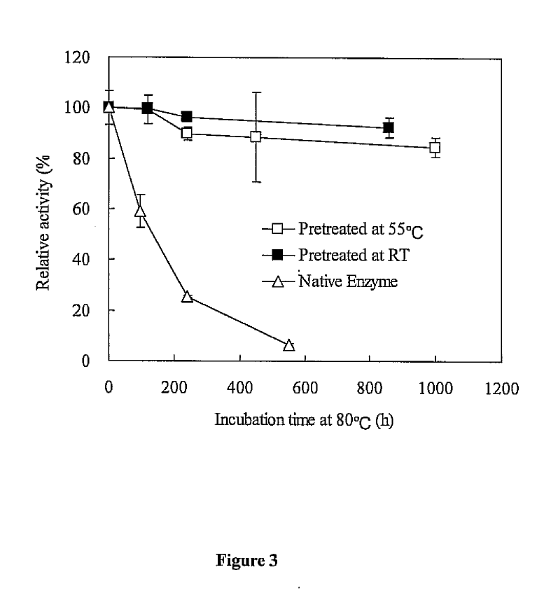

As can be seen in FIG. 3 pretreatment of α-chymotrypsin results in a surprising enhancement of the enzyme thermal stability at 80° C. at which no significant difference for the single methods (drying at RT or 55° C.) can be observed. Over a period of 1000 hours only 15% of the initial activity is lost despite the high temperature of 80° C., whereas the native in wet and dry state lost activity much faster.

Stability of Pretreated (Dry) Hydrogel-Entrapped α-CT in Methanol

After pretreatment, the stability of hydrogel-entrapped α-CT was investigated in methanol. A single pretreated hydrogel disc from the same batch was incubated in 10 ml of pure methanol in a 20-mL screw-capped vial. The native enzyme and non-pretreated hydrogel discs served as comparisons. The hydrogel discs were removed from the solvent at specific time to determine the residue activity (assay described above).

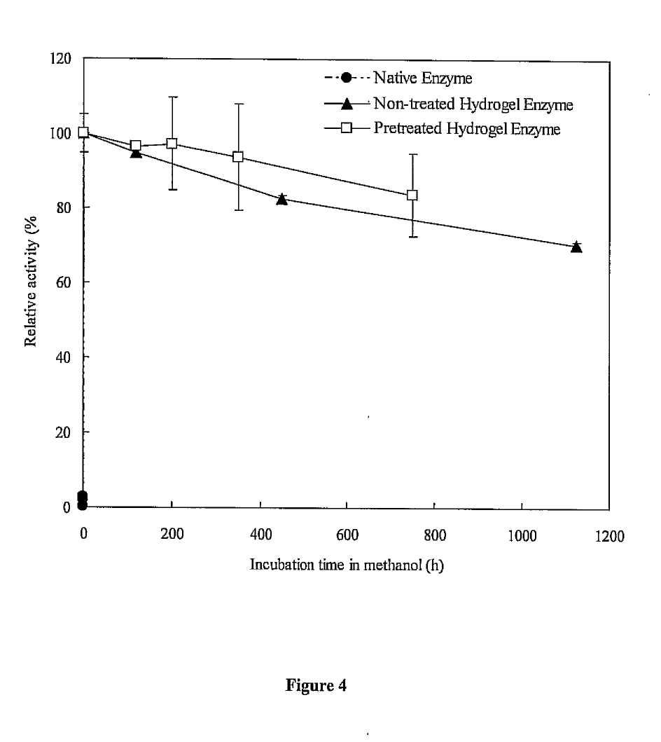

Gel-entrapped α-CT also showed greatly enhanced stability in organic solvent as detailed in FIG. 4. The half-life of dry gel α-CT was about 140 days in methanol, a surprising 10⁵-fold enhancement over native α-CT.

Transesterification Activity of Native and Pretreated Hydrogel α-CT

The transesterification activity of native α-CT in organic solvents was measured at room temperature in hexane or isooctane containing APEE (concentration ranged from 2.5 to 30 mM) and 0.5 M n-PrOH. Typically, 5 mg of native CT powder was added to 10 mL of reaction solution to initiate the reaction. During reaction at 200 rpm, aliquots of 200 μl from the reaction solution was periodically removed by filtration using a 0.22 μm PTFE syringe filter following centrifugation for 5 minutes at 13,000 rpm. A volume of 100 μl of supernatant was used for gas chromatograph analysis (GC method listed below).

For hydrogel-entrapped α-CT, one piece of dried hydrogel disc was added into 10 ml of hexane or isooctane containing APEE (concentration ranged from 2.5 to 30 mM) and 0.5 M n-PrOH. The reaction was shaken at 200 rpm, while aliquots of 200 μl were taken periodically and centrifuged for 5 minutes at 13,000 rpm. A volume of 100 μl of supernatant was used for gas chromatograph analysis.

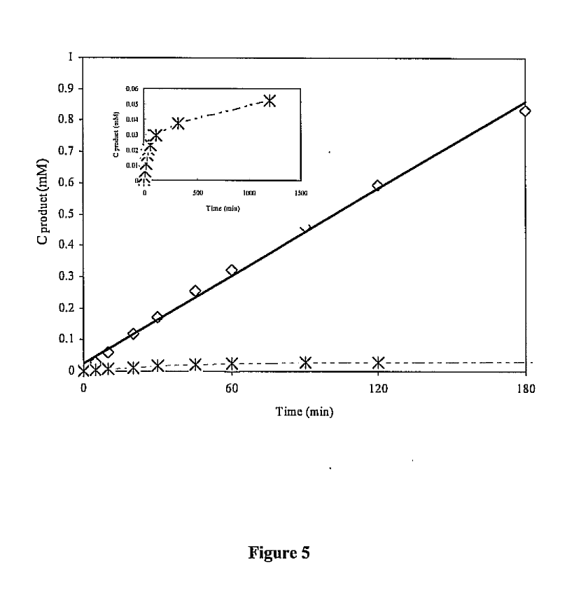

The product concentration was monitored by using a gas chromatograph equipped with a FID detector and a RTX-5 capillary column (0.25 mm×0.25 μm×10 m, Shimadzu). A temperature gradient from 100 to 190° C. at a heating speed of 20° C./min, followed by 5-min retention at 190° C. was used. The injection temperature column was kept at 210° C. whereas the detector temperature was 280° C. The initial reaction rate of the formation of n-acetyl-L-phenylalanine propyl ester (APPE) was calculated. As shown in FIG. 5 a water content of 1% resulted in an initial transesterification rate of 0.8 μmol APPE/min/mg enzyme, which is around 13 folds higher compared to the specific activity of the native enzyme.

Water Content Effects on the Activity

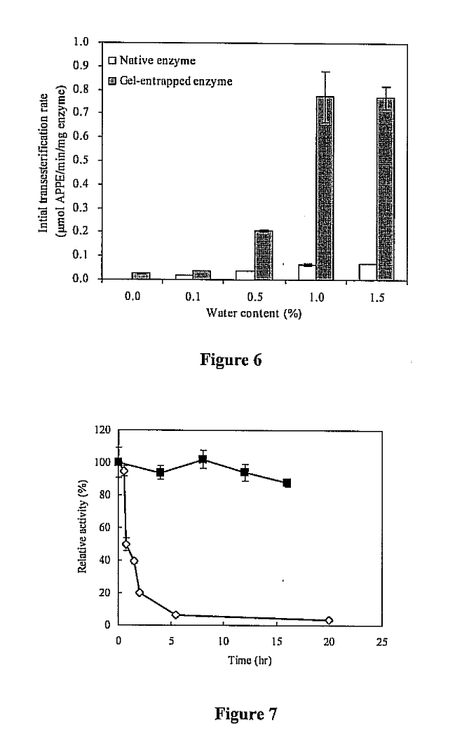

The activity of gel-confined α-CT was examined in organic solvent with varying water amount ranging from 0 to 1.5 v/v % in n-hexane. Native α-CT powder suspended in hexane with the same water content served as a control. As detailed in FIG. 6 the water content has a significant impact on the activity of the gel-confined α-CT, with a maximum reached with 1% water. At this water content in comparison to native α-CT powder suspended in hexane, gel-confined enzyme showed over 3 orders of magnitude of enhancement for transesterification activity when no additional water was added to the reaction. At 0.1% and 0.5% water content the gel-entrapped enzyme showed only 2-fold and 4.5-fold enhanced activity compared to the native enzyme. This observation may be due to the water-competing effects between hydrogel and enzyme molecules and the mass transfer limitation in the gel for both substrate and product. There was no observed increase in the initial reaction rate when water content was increased from 1.0% to 1.5%, inferring that mass transfer played a minor role when a gel was hydrated beyond the 1.0% threshold water content.

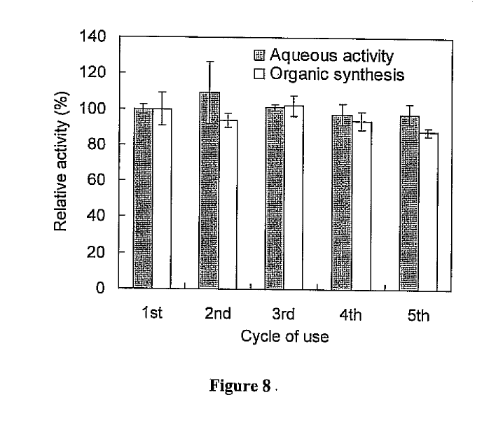

Referring to FIG. 7 it can be seen that not only the activity but also the operational stability of gel confined α-CT in the organic solvent was improved. The reaction rate of native enzyme decreased quickly over time: The native enzyme lost more than 90% of the initial activity within the first 5 hours, whereas the gel-confined enzyme showed only an activity loss of 10% in a period of 16 hours. It was also shown that the gel-confined enzyme can be reused for at least 5 cycles without significant activity loss for both aqueous hydrolytic and organic transesterification reactions, as shown in FIG. 8.

1. A bioactive composition comprising:
a porous hydrogel matrix;
at least one protein immobilized in the porous hydrogel matrix forming a hydrogel-protein composite that is stable in an organic solvent.

2. The bioactive composition of claim 1 wherein the hydrogel protein composite has a half-life at least 1000 times longer than the half-life of a free digestive protein counterpart in the organic solvent.

3. The bioactive composition of claim 1 wherein the hydrogel protein composite maintains an activity in the organic solvent.

4. The bioactive composition of claim 1 wherein the hydrogel protein composite has an activity 1000 times greater than a free digestive protein counterpart in the organic solvent.

5. The bioactive composition of claim 1 wherein the hydrogel protein remains biologically active at elevated temperatures.

6. The bioactive composition of claim 1 wherein the protein remains biologically active after exposure to a temperature up to 110° Celsius.

7. The bioactive composition of claim 1 wherein the protein is selected from the group consisting of protease, amylase, cellulose, lipase, peroxidase, tyrosinase, glycosidase, nuclease, aldolase, phosphatase, sufatase, dehydrogenase, and lysozyme and combinations thereof.

8. The bioactive composition of claim 1 further including a biochemically active agent selected from the group consisting of antibody, nucleic acid, fatty acid, hormone, vitamin, mineral, structural protein, enzymes, and therapeutic agents including histamine blockers and heparin.

9. The bioactive composition of claim 1 wherein the hydrogel matrix includes a water content of from 0 to 5 weight percent of the hydrogel matrix,

10. The bioactive composition of claim 1 wherein the digestive protein is between about 0.2 to 4.5 dry weight percent of the hydrogel matrix.

11. A process for stabilizing a bioactive composition comprising:
forming hydrogel matrix pores around protein molecules; and
reducing a water content within the hydrogel matrix pores forming a hydrogel-protein composite that is stable in an organic solvent.

12. The process of claim 11 wherein the step of forming hydrogel matrix pores around protein molecules includes the steps of dissolving the protein in deionized water at a desired concentration, dissolving a prepolymer in deionized water at a desired concentration, mixing the dissolved protein and dissolved prepolymer in a desired ratio, and initiating a polymerization of the prepolymer.

13. The process of claim 12 wherein the step of initiating the polymerization includes at least one step selected from: adding a cross linking agent, adding an initiator, and adjusting a temperature of the mixture of the dissolved protein and dissolved prepolymer.

14. The process of claim 11 wherein the ratio of dissolved prepolymer is from 20-28 percent by weight in relation to the total volume.

15. The process of claim 11 wherein the step of reducing a water content within the hydrogel matrix pores comprises heating the hydrogel matrix to a temperature of from 20 to 110 degrees Celsius for a time period of from 24 hours to seven days.

16. The process of claim 15 wherein the step of reducing a water content within the hydrogel matrix pores comprises heating the hydrogel matrix to a temperature of from 20 to 80 degrees Celsius for a time period of 24 hours followed by air drying at room temperature for one week.

17. The process of claim 15 wherein the step of reducing a water content within the hydrogel matrix pores comprises heating the hydrogel matrix to a temperature of from 20 to 55 degrees Celsius for a time period of 24 hours followed by air drying at room temperature for one week.

18. The process of claim 11 wherein the step of reducing a water content within the hydrogel matrix pores reduces pore volumes from 15 to 21 percent compared to a wet gel volume.

19. The process of claim 11 wherein the step of reducing a water content within the hydrogel matrix pores reduces pore volumes and allows a substrate to enter the pores allowing a reaction between the protein and the substrate.
